(19) United States
(12) Patent Application Publication
(10) Pub. No.: US 2015/0133934 A1
(43) Pub. Date: May 14, 2015

(54) Implant

(71) Applicant: The University of Nottingham, Nottinghamshire (GB)

(72) Inventors: Reda Felfel, Nottinghamshire (GB); Chris Rudd, Nottinghamshire (GB); Andrew Parsons, Nottinghamshire (GB); Ifty Ahmed, Nottinghamshire (GB)

(21) Appl. No.: 14/398,517

(22) PCT Filed: May 2, 2013
(86) PCT No.: PCT/GB2013/051150

§ 371 Date: Nov. 3, 2014

(30) Foreign Application Priority Data
May 4, 2012 (GB) 1207882.0

(51) Int. Cl. B29C 51/00; A61B 17/80; A61B 17/86

(52) U.S. Cl. CPC B29C 51/002; A61B 17/86; A61B 17/80; A61B 2017/00526
USPC 606/60; 264/322

(57) ABSTRACT

A method of manufacture of an at least partially bioresorbable implant, the method comprising the steps of: providing an at least partially bioresorbable precursor containing a polymeric material; and forming an implant by forging the precursor. An at least partially bioresorbable forged implant containing a polymeric material.

The present invention relates to a method of manufacture of fixation devices, more particularly to a method of manufacture of fixation devices such as screws, pins, rods and plates for use in human or animal bodies, e.g. in treating bone fractures. The present invention also relates to such fixation devices.

It is known to use screws, pins, rods and plates in treating bone fractures. For instance, a screw and plate system is the most commonly used system for internal fixation of bone fragments to stabilise the bone fragments.

Metallic fixation devices, e.g. plates and screws, are known and, commonly, may be made from titanium, cobalt alloys and stainless steel. However, while metallic fixation devices may perform well mechanically, there are disadvantages associated with their use. Such disadvantages may include the requirement for removal operations, allergenic response, corrosion, MRI interference and long-term infection issues. Also, metallic implants, e.g. fixation devices, are known to cause bone atrophy as a result of the mismatch between the mechanical properties of bone and metals (causing stress shielding).

The use of biocompatible, bioresorbable materials in fixation devices has potential to reduce and potentially eliminate the drawbacks associated with metals. However, the initial mechanical properties of biocompatible, bioresorbable polymers such as poly lactic acid (PLA) typically may not be adequate to fix bone fractures except in relatively low load bearing areas such as the face or skull (i.e. craniomaxillofacial).

A first aspect of the invention provides a method of manufacture of an at least partially bioresorbable implant, the method comprising the steps of: providing an at least partially bioresorbable precursor containing a polymeric material; and forming an implant by forging the precursor. Typically, the implant may be a fixation device.

Preferably, forging may be carried out at a temperature between the glass transition temperature (T_{g}) and the crystallisation temperature (T_{c}) for the polymeric material. The person skilled in the art will be able to select an appropriate forging temperature for a given polymeric material. The forging process may be a cold forging process.

Typically, forging may comprise placing the precursor in a cavity of a mould.

In an embodiment, the precursor may comprise a composite containing fibres and/or particles embedded in a polymeric matrix.

Advantageously, the fibres and/or particles may provide mechanical reinforcement within the fixation device. The fibres and/or particles may improve the mechanical strength of the fixation device. Advantageously, the mechanical properties of the fixation device may be such that the fixation device may be used in treating or fixing bone fractures even in load bearing areas of the body. Fibres may improve the mechanical stiffness as well as the mechanical strength of the fixation device.

The fibres and/or particles may be substantially uniform in size. Alternatively, a plurality of different sizes of fibres and/or particles may be used in the manufacture of a given fixation device.

The proportion, e.g. volume fraction, of fibres and/or particles may vary, preferably in a predetermined manner, or may be substantially uniform within the fixation device.

Preferably, the fibres may be randomly oriented within the precursor and/or may be aligned in one or more directions.

Preferably, some of the fibres may be aligned in one or more directions and some of the fibres may be randomly oriented.

The proportion of fibres which are randomly oriented may be up to 100%, up to 70%, up to 50%, or up to 30%, by volume of the fibres and/or particles within the precursor and/or the fixation device.

The precursor may comprise a first region or layer in which the fibres are aligned in one or more directions and a second region or layer in which the fibres are randomly oriented.

Preferably, the fibres may be oriented in a single direction (i.e. unidirectionally) or in two distinct directions (i.e. bidirectionally).

In an embodiment, at least some of the fibres and/or particles may be bioresorbable.

Preferably, the fibres and/or particles may comprise glass fibres and/or particles. The fibres and/or particles may comprise phosphate-based glass fibres or particles. Phosphate-based glass fibres and/or particles may be especially preferred, due to their good biocompatibility, osteoconductivity, mechanical properties and also due to their being fully bioresorbable.

A suitable glass composition may be 40P₂O₅-24MgO-16CaO-16Na₂O-4Fe₂O₃ in mol %. Conveniently, this glass composition may be denoted P40.

The fibres may be at least 2 mm long and/or up to 200 mm long. The fibres may have a diameter of up to 30 μm, e.g. around 15 μm.

The fibres may be produced by any suitable process, e.g. melt-draw spinning.

In an embodiment, the polymeric material, e.g. the polymeric matrix, may be at least partially bioresorbable. For instance, the polymeric material may comprise one or more of poly lactic acid (PLA), poly glycolic acid (PGA) and polycaprolactone (PCL) or copolymers thereof.

Preferably, the fixation device may be substantially or even completely bioresorbable. Thus, in some preferred embodiments, the fibres and/or particles and the polymeric matrix may both be bioresorbable.

Advantageously, in use, the fixation device may gradually lose its strength over time and be designed to be completely absorbed after bone healing has occurred, without causing any deleterious effects.

Conveniently, the precursor may be provided in the form of a bar.

In an embodiment, the method may comprise the preliminary step of manufacturing or providing a master plate and cutting the precursor from the master plate. The master plate may be manufactured using a film stacking process.

The fixation device may have the form of a screw, e.g. a cortical screw or a cancellous screw, a pin, a rod or a plate. The screw may comprise a fully or partially threaded shaft. The screw may comprise a screw head or the screw may be a headless screw.

Cortical and cancellous screws are named according to the type of bone they are designed for. Typically, cortical screws may have a smaller pitch and thread depth than cancellous screws. In general, cortical screws are stronger than cancellous screws with the same outer diameter as their core diameter is larger. Three kinds of cortical screw are currently used: unicortical, bicortical and lag screws. Unicortical and bicortical screws are fully threaded screws; unicortical screws (short screws) fasten to a single layer of cortical bone, whilst bicortical screws are longer and able to engage both cortical layers (i.e. passing completely through the bone). Lag screws are longer and only threaded at one end, in order to fix only to the far cortex. Cancellous screws are designed to be fixed to the metaphysis of long bones where cancellous bone is abundant, utilising a larger surface area to spread the load in a considerably weaker bone structure.

The screw may have a rough thread surface. Advantageously, the rough thread surface may improve, in use, the interlock between the screw and bone.

Preferably, the particles or fibres may comprise at least 10% and/or up to 60% by volume of the precursor and/or the fixation device. More preferably, the particles or fibres may comprise at least 20% and/or up to 50%, e.g. around 30%, by volume of the precursor and/or the fixation device.

The use of forging, e.g. cold forging, to form the fixation device may offer several advantages. First, significant changes in the composite structure typically do not occur, i.e. the structure of the composite bar is substantially preserved in the fixation device. Furthermore, no additional processing or machining may be required. For instance, a fixation device in the form of screw may be made by forging from a composite bar without the need for subsequent machining, e.g. to form a screw thread. Machining, e.g. to form a screw thread, may damage or break fibres or particles within the matrix, thereby potentially weakening the fixation device. For instance, machining a threaded portion of a screw, in which the shaft contains unidirectionally aligned fibres extending in a lengthwise direction along the shaft may damage or break the fibres. Advantageously, such damage may be substantially reduced or may not even occur when forging is used.

In addition, since much less, or even no, additional processing or machining may be required, the method of manufacture may be simpler and/or quicker and/or more reliable and/or more cost-effective.

A second aspect of the invention provides an at least partially bioresorbable forged implant, preferably a fixation device, containing a polymeric material.

In an embodiment, the forged implant, e.g. fixation device, may comprise fibres and/or particles embedded in a polymeric matrix.

The fixation device may be a screw. The screw may have a rough thread surface.

A third aspect of the invention provides an at least partially bioresorbable screw having a rough thread surface. The screw may be manufactured by forging. The screw may contain a polymeric material. Typically, the screw may comprise fibres and/or particles embedded in a polymeric matrix.

A fourth aspect of the invention provides a system for internal fixation of bone fragments in humans or animals comprising at least one at least partially bioresorbable forged implant, e.g. fixation device, containing a polymeric material. In a preferred embodiment, the system may comprise a screw and, optionally, a plate.

A fifth aspect of the invention provides a method of fixing a bone fracture comprising the use of a fixation device or system according to the invention.

Advantageously, in some embodiments, an implant, e.g. a fixation device, according to the invention may be made from biocompatible materials with initial mechanical properties matching or surpassing those for cortical bone. In use, the fixation device may gradually lose its strength over time and be completely absorbed after bone healing has occurred, without causing any deleterious effects.

In order that the invention may be well understood it will now be described, by way of example only, with reference to the accompanying drawings, in which:

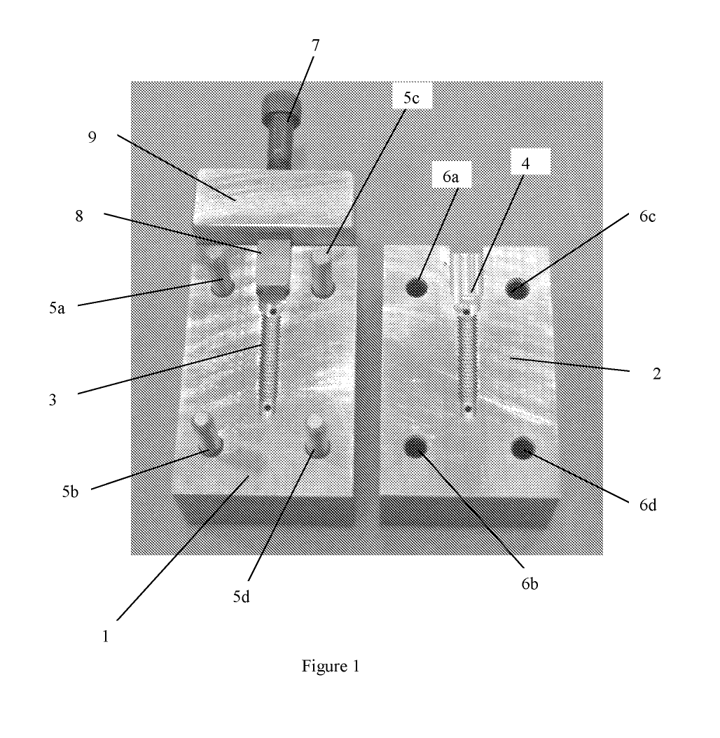

FIG. 1 shows a mould for use in the method of manufacture according to the invention;

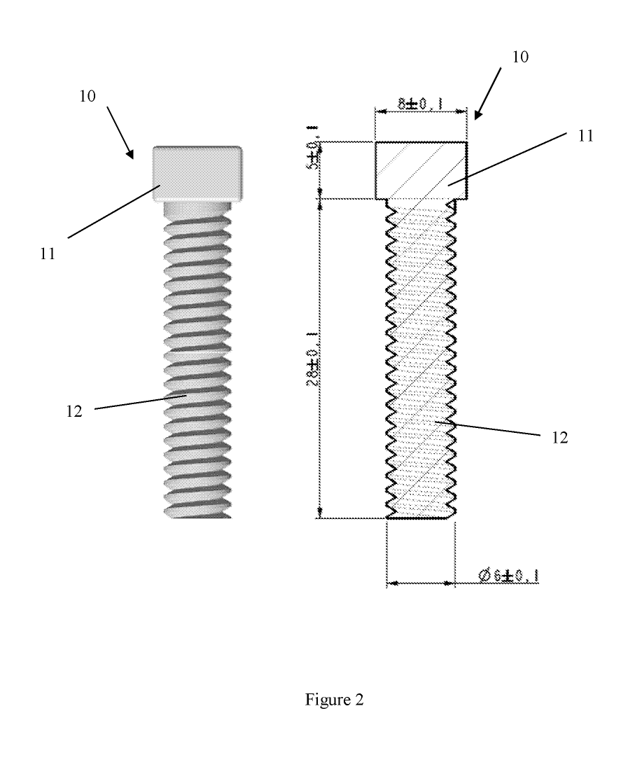

FIG. 2 shows a square headed screw manufactured in accordance with the invention;

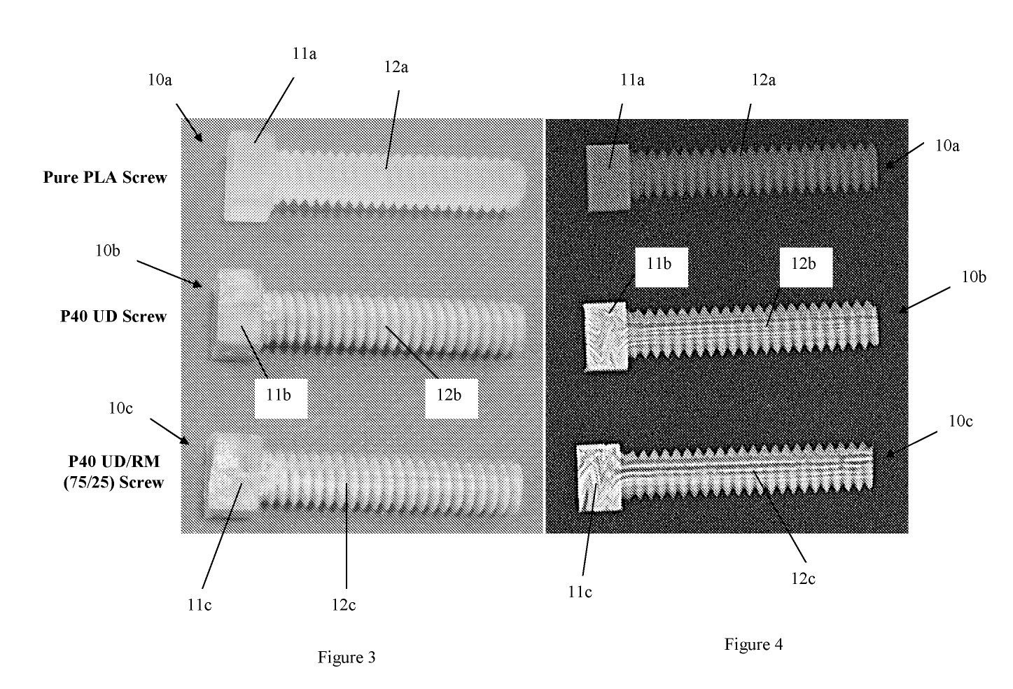

FIG. 3 shows photographs of three examples of square headed screws manufactured in accordance with the invention;

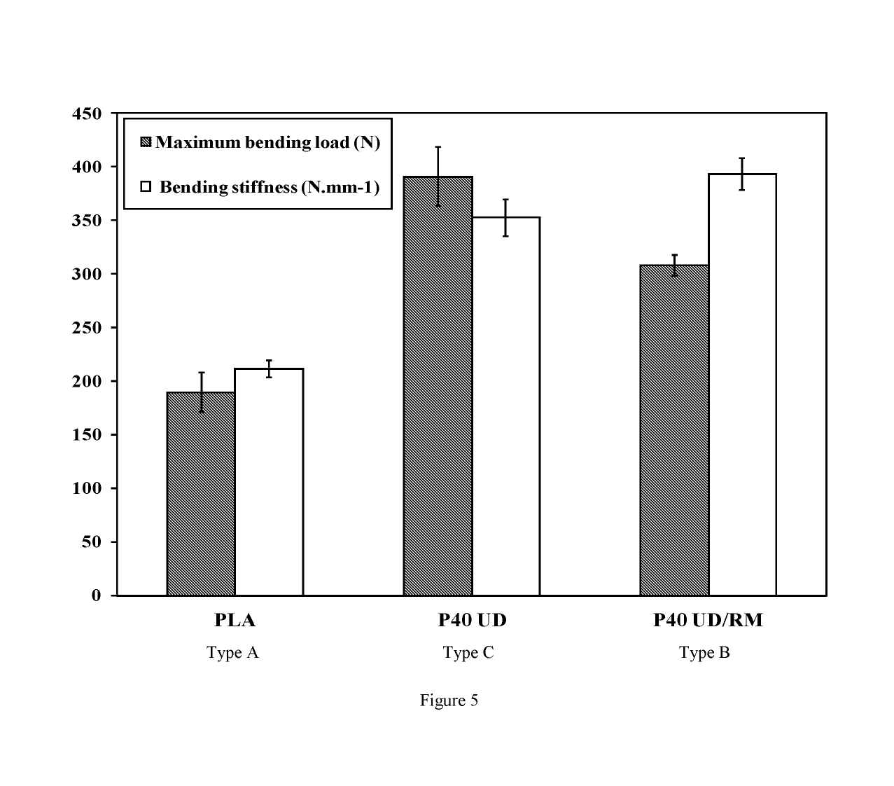

FIG. 4 shows x-ray photographs of the screws shown in FIG. 3;

FIG. 5 is a bar chart showing flexural load to failure and stiffness for three types of screw manufactured in accordance with the invention;

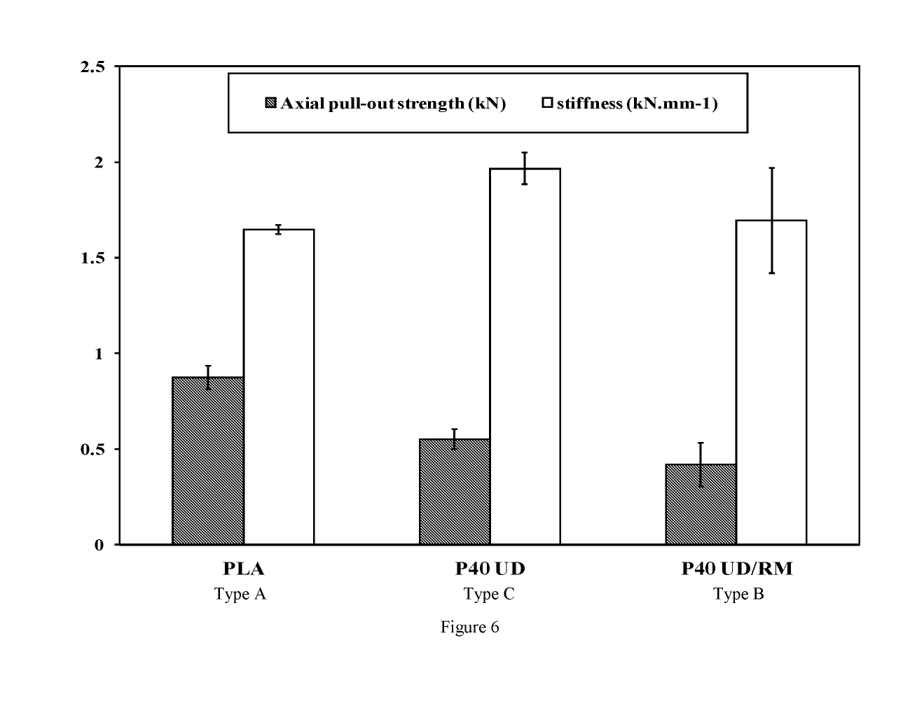

FIG. 6 is a bar chart showing axial pull-out strength and stiffness for three types of square headed screw manufactured in accordance with the invention;

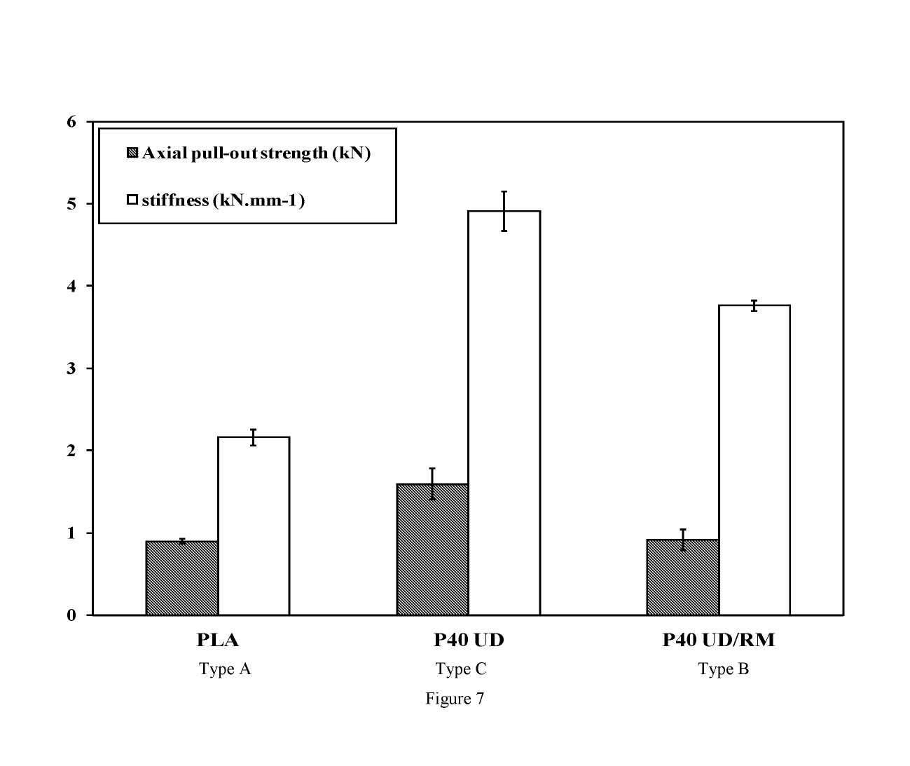

FIG. 7 is a bar chart showing axial pull-out strength and stiffness for three types of headless screw manufactured in accordance with the invention;

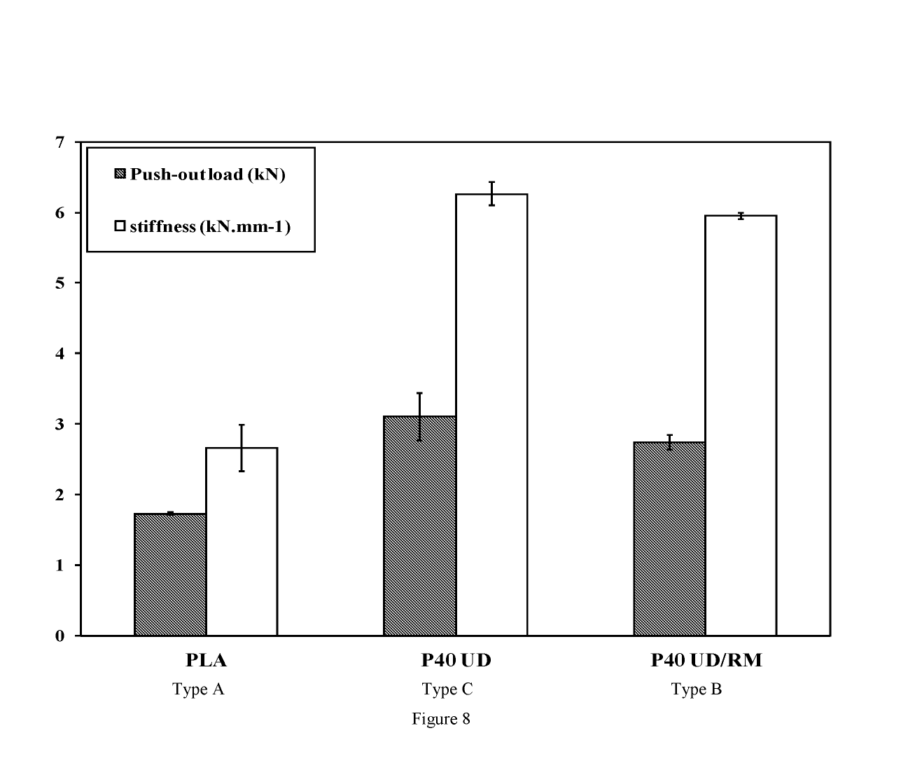

FIG. 8 is a bar chart showing push-out load to failure and stiffness for three types of square headed screw manufactured in accordance with the invention;

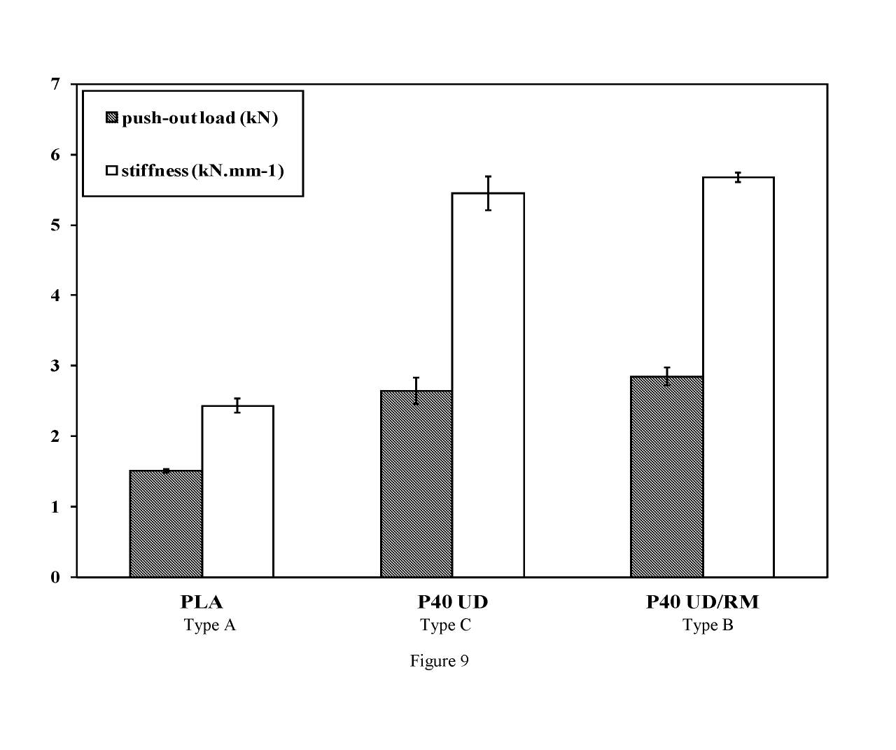

FIG. 9 is a bar chart showing push-out load to failure and stiffness for three types of headless screw manufactured in accordance with the invention;

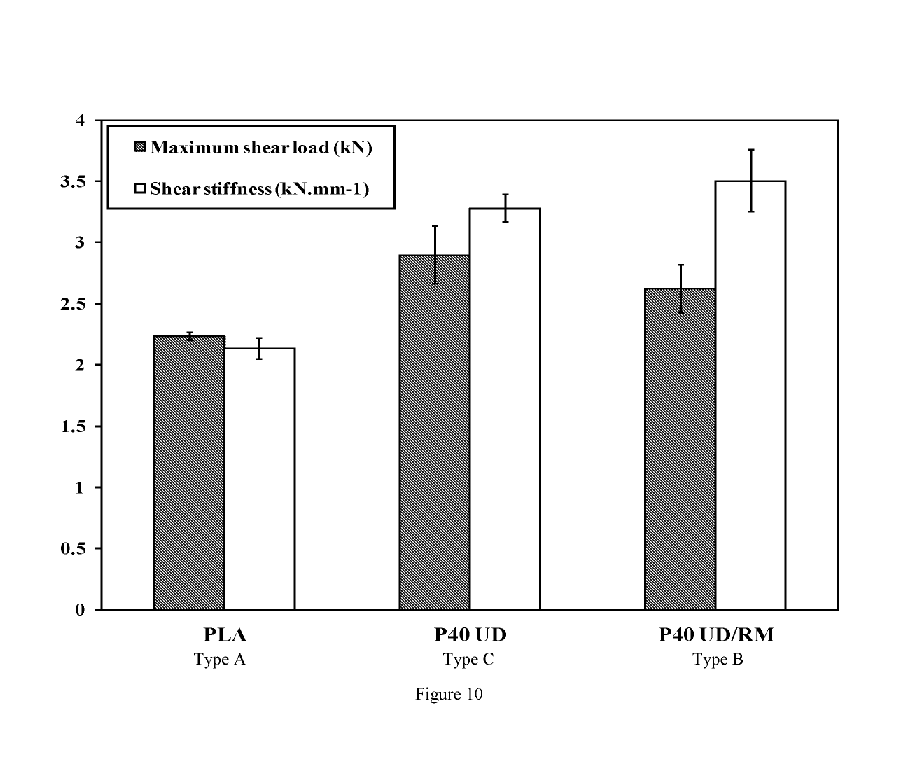

FIG. 10 is a bar chart showing shear load to failure and stiffness for three types of screw manufactured in accordance with the invention;

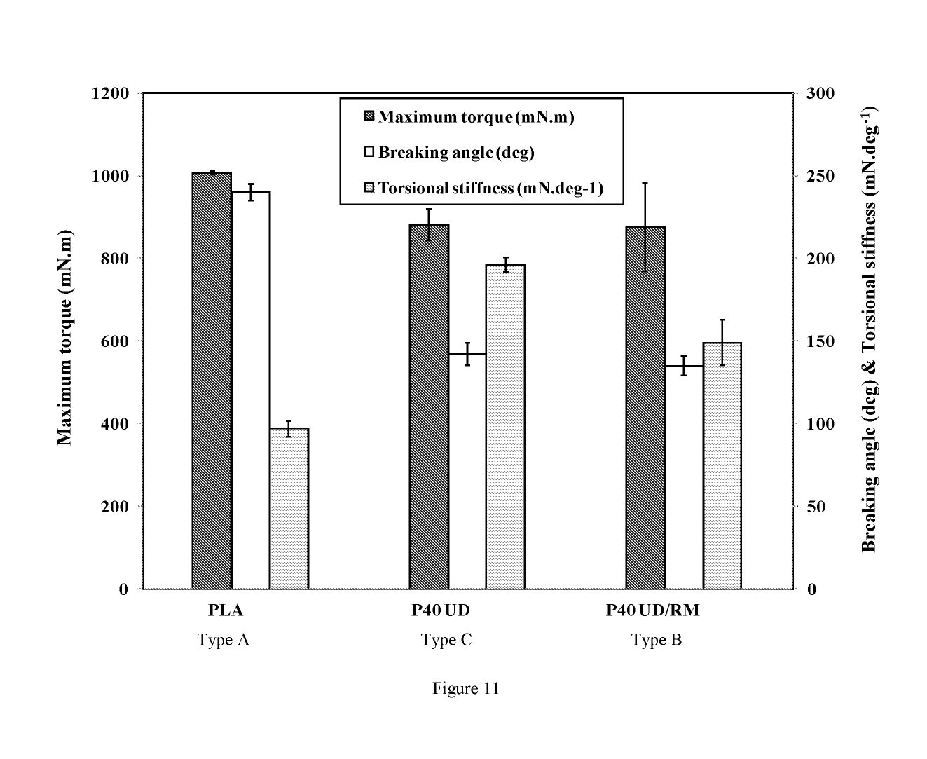

FIG. 11 is a bar chart showing maximum torque, breaking angle and torsional stiffness for three types of screw manufactured in accordance with the invention;

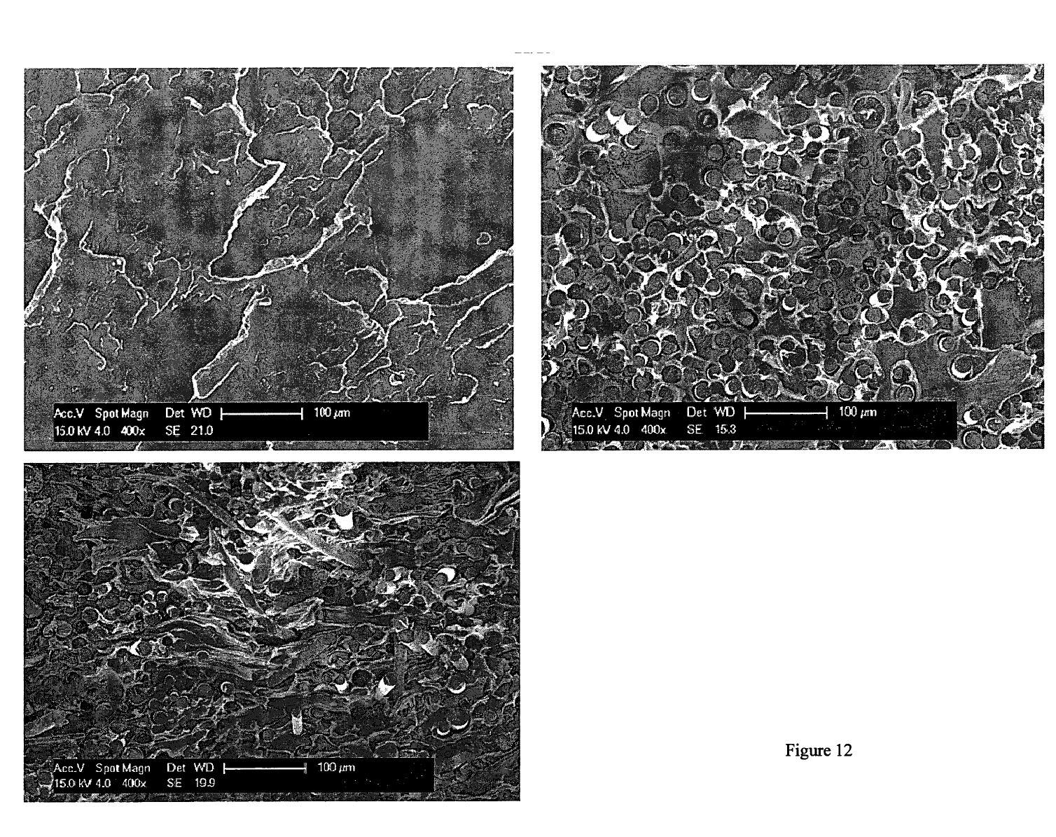

FIG. 12 shows scanning electron microscope (SEM) micrographs of a fractured surface after a flexural test of three types of screw manufactured in accordance with the invention;

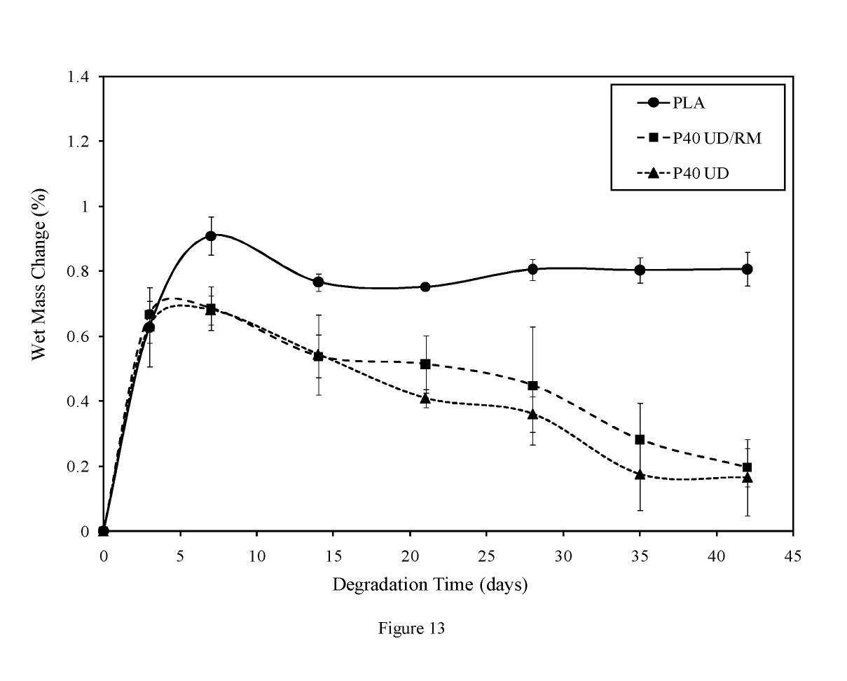

FIG. 13 is a graph showing the percentage change in wet mass over time for three types of screw manufactured in accordance with the invention immersed in phosphate buffered saline (PBS) solution at 37° C.;

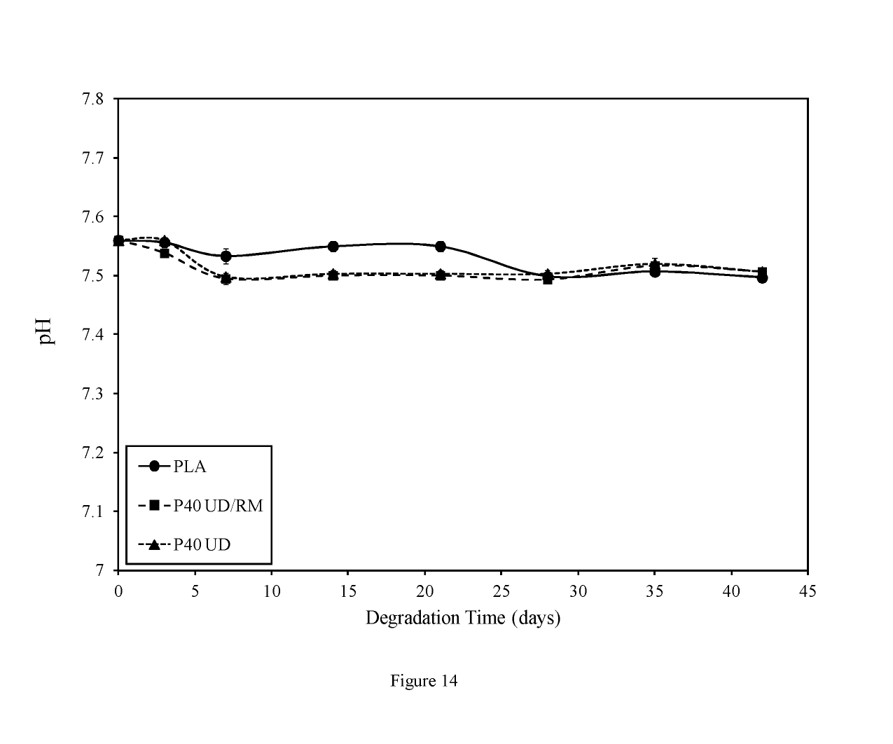

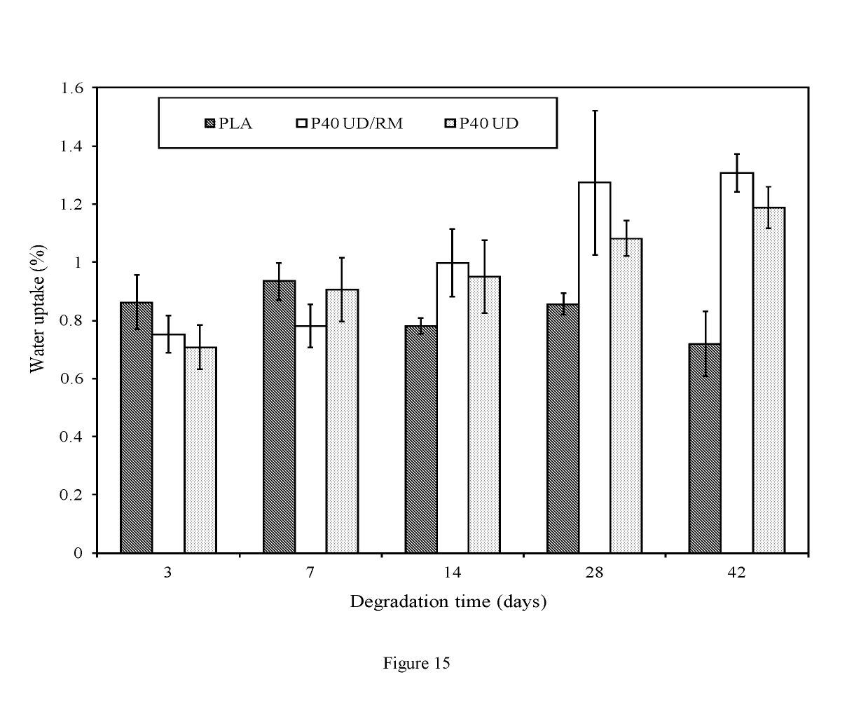

FIG. 14 is a graph showing pH change over time for three types of screw manufactured in accordance with the invention immersed in phosphate buffered saline (PBS) solution at 37° C.;

FIG. 15 is a bar chart showing percentage change in water uptake over time for three types of screw manufactured in accordance with the invention immersed in phosphate buffered saline (PBS) solution at 37° C.;

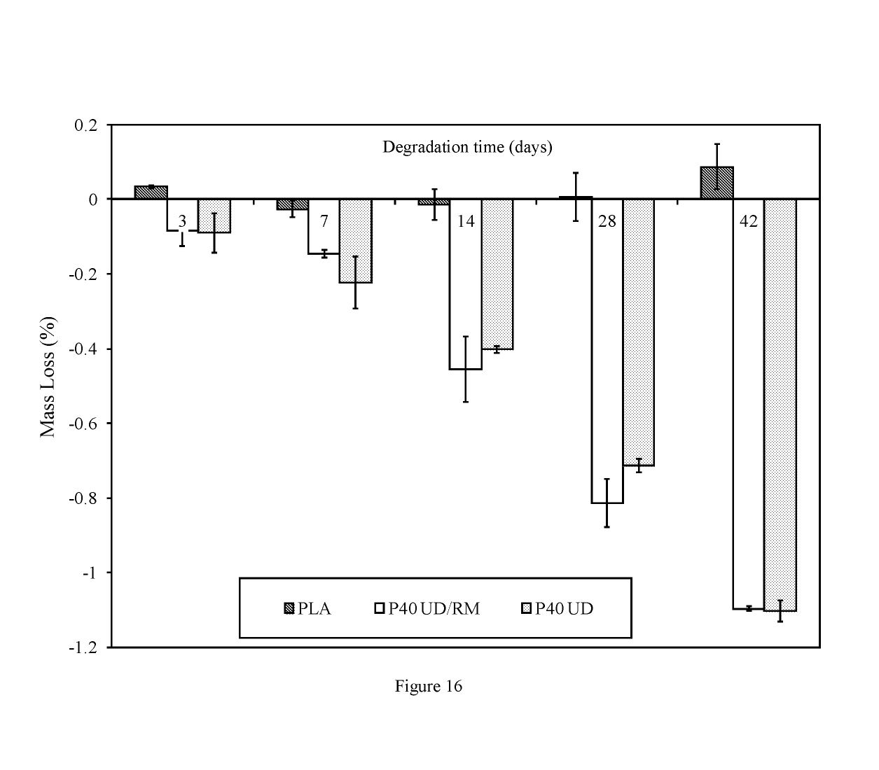

FIG. 16 is a bar chart showing percentage change in mass over time for three types of screw manufactured in accordance with the invention immersed in phosphate buffered saline (PBS) solution at 37° C.;

Phosphate-based glass fibres (PGF) and mats were prepared as follows. The glass was produced with the composition 40P₂O₅-24MgO-16Ca)-16Na₂O-4Fe₂O₃ in mol %—denoted as P40. Continuous fibres with ˜15 μm diameter were produced by melt-draw spinning at ˜1100° C. and ˜1600 rpm. The fibres were annealed for 90 minutes at 5° C. below the glass transition temperature T_{g} (T_{g}=479° C.) prior to use.

Random non-woven fibre mats (RM) were prepared from 20 mm chopped fibres by dispersion in a Cellosize (hydroxyethyl cellulose) and then rinsed with deionised water to remove any residual binder before being dried at 50° C. for 30 minutes.

Unidirectional (UD) fibre mats were produced from 110 mm long fibre bundles aligned and bound by Cellosize solution. The UD mats were also rinsed with deionised water to remove any residual binder before being dried at 50° C. for 30 minutes.

Precursors were prepared as follows. Poly lactic acid (PLA) (Resin 3251-D NatureWorks® average Mw ˜90,000-120.000, polydispersity index (PDI)=1.636, melting temperature Tₘ=170.9° C., T_{g}=61.3° C.) films (approx 0.2 mm thick) were prepared via compression moulding at 210° C. and 3 bar. The PLA pellets and RM and UD fibres were dried in a vacuum oven at 50° C. for 48 hours before use.

Both UD/RM and UD composite precursors were prepared via a film stacking process. The films were stacked alternately with UDs and RMs in a 110 mm (width)×110 mm (length)×4.5 mm (thickness) mould placed between two metallic plates. This stack was then heated in the press for 15 minutes at 210° C. and pressed for 15 minutes at 38 bar. The plates were transferred to a second press for cooling to room temperature at 38 bar for 15 minutes.

Screws were prepared by forging composite bars. The temperature for forging should be between the glass transition temperature (T_{g}) and the crystallisation temperature (T_{c}) for the polymeric material or matrix to avoid further crystallisation in the specimens. PLA has a T_{g} of around 60° C. and a T_{c} of around 112° C. For example, a suitable forging temperature for forging implants comprising PLA may be around 80° C.

The laminated composite and pure PLA master plates produced as described above were cut into 40 mm length×6.5 mm width×4.5 mm height pieces or bars using a band saw, which were then placed into a cavity of a mould (see FIG. 1) specially made to manufacture screws.

Referring to FIG. 1, the mould comprises a first block 1 and a second block 2. In a surface of the first block 1 there is a first recess 3 extending from an edge of the surface and having a portion with the shape of a screw cut in half along its length. The first block 1 is provided with four rods 5a, 5b, 5c, 5d, each rod 5a, 5b, 5c, 5d being located towards a corner of and protruding vertically from the surface of the first block 1 in which the first recess 3 is formed. Each rod 5a, 5b, 5c, 5d is substantially cylindrical. As shown in FIG. 1, a compression member 8 is housed partially within and extends from a third block 9 into the first recess 3. A screw 7 is operably connected to the compression member 8. In use, the screw 7 is operable to drive the compression member 8 into and out of the first recess 3 in a longitudinal direction. The compression member 8 cannot be driven into the portion of the first recess 3 that has the shape of a screw cut in half along its length.

In a surface of the second block 2 there is a second recess 4 extending from an edge of the surface and having a portion with the shape of a screw cut in half along its length. The second block 2 is provided with four holes 6a, 6b, 6c, 6d, each hole 6a, 6b, 6c, 6d being located towards a corner of and extending vertically into the surface of the first block 2 in which the second recess 4 is formed.

In use, the first block 1 and the second block 2 are brought together such that the rods 5a, 5b, 5c and 5d are received in the holes 6a, 6b, 6c and 6d, respectively. The first recess 3 and the second recess 4 are aligned and together provide the cavity into which the bars were placed.

The mould was heated in a press for 10 minutes at 100° C. and then pressed for 30 seconds at 3 bar. Then, the screw 7 was screwed in to cause the compression member 8 to compress the end of the composite bar into a square screw head.

Afterwards, the mould was transferred to a cold press for cooling under 3 bar for 5 minutes.

Referring to FIG. 2, an example of a finished screw 10 has a square head 11 and a fully threaded shaft 12. The screw 10 has an overall length of 33 mm; the shaft 12 is 28 mm long and the head 11 makes up the remainder of the overall length of the screw. The head 11 is 8 mm across. The threaded shaft 12 has an outer diameter of 6 mm and a core diameter of 4.75 mm.

FIGS. 3 and 4 show three examples of screws manufactured as described above: a pure PLA screw 10a; a P40 UD screw 10b; and a P40 UD/RM (70/25) screw 10c. The screws 10a, 10b and 10c each have a square head 11a; 11b; 11c and a fully threaded shaft 12a; 12b; 12c and have similar dimensions to the screw 10 generally illustrated in FIG. 2. FIG. 3 shows optical photographs of the screws 10a, 10b, 10c. FIG. 4 shows x-ray photographs of the screws 10a, 10b, 10c. The fibres can be seen in the x-ray photograph of the P40 UD screw 10b and the P40 UD/RM (70/25) screw 10c.

The fibre volume and mass fractions of three types of screw manufactured as described above are shown in Table 1. The values were obtained using the matrix burn off method, according to the ASTM standard test method (ASTM D2584-94).

TABLE 1

| | Fibre mass | Fibre volume |
|---|---|---|
| Screw | fraction (%) | fraction (%) |
| PLA | — | — |
| Pure PLA screw (hereinafter “Type A”) | | |
| P40 UD/RM | 50 ± 3 | 31 ± 3 |
| P40 glass fibre reinforced PLA composite | | |
| screws: 70% unidirectional fibre/30% | | |
| random mat (hereinafter “Type B”) | | |
| P40 UD | 50 ± 2 | 31 ± 2 |
| P40 glass fibre reinforced PLA composite | | |
| screws: 100% unidirectional fibre | | |
| (hereinafter “Type C”) | | |

Mechanical tests and in vitro degradation tests were carried out to characterise these three types of forged screws manufactured by the method described above and to check that they might be fit for purpose. In at least some tests, headless screws were tested as well as square headed screws.

For convenience and clarity, the PLA screws that were tested will be referred to hereinafter as “Type A”, the P40 UD/RM screws that were tested will be referred to hereinafter as “Type B” and the P40 UD screws that were tested will be referred to hereinafter as “Type C”.

The maximum flexural load and stiffness for the three types of screw were evaluated by flexural (three-point bending) tests using a Hounsfield Series S testing machine at room temperature (˜20° C.). A crosshead speed of 5 mm/min and a 1 kN load cell was used. The support span was 20 mm and radii for loading applicator and supports were 2.5 mm. The maximum flexural load was the maximum value recorded during the test and the stiffness was the maximum gradient in a load-deflection plot. The measurements were conducted in triplicate (n=3).

With reference to FIG. 5, it can be seen that the flexural load (maximum bending load) and stiffness (bending stiffness) values for Type C (P40 UD) screws were approximately double that for Type A (pure PLA) screws. For Type B screws, the maximum load and stiffness were approximately 75% and 100% higher than for the Type A screws (approximately 190 N and approximately 200N·mm⁻¹) respectively.

The axial pull-out strength and stiffness were determined using an Instron 5969 according to the standard ASTM F 2502-05 with modified setup. A crosshead speed of 5 mm/min and a 25 kN load cell was used. During the tests, the screw was inserted to a depth of 60% (˜15 mm) of the total length of the thread. The measurements were carried out in triplicate (n=3). The axial pull-out strength was determined as the maximum load reached during the test and the stiffness was the maximum gradient in a load-deflection plot. The type of failure was also reported.

Axial pull-out strength and stiffness for PLA (Type A) and composite (Type B and Type C) screws are shown in FIG. 6. The pull-out strength and stiffness for Type A screws were ˜0.9 kN and ˜1.7 kN·mm⁻¹. Type C and Type B composite screws had slightly higher pull-out stiffness, but lower pull-out strength than Type A screws.

The pull-out test was applied also for headless screws in a thread to thread test after insertion of 30% (7.5 mm) of the overall thread length into each tapped jaw. Furthermore, a push-out test was conducted on square headed and headless screws using the variables mentioned previously.

Referring to FIG. 7, it can be seen that the axial pull-out strength for headless PLA (i.e. Type A) screws was very similar to that of the screws with heads (see FIG. 6) and their stiffness was slightly higher (˜2 kN·mm⁻1). For Type C headless screws, the pull-out strength and stiffness increased by ˜80% and ˜130% in comparison with the Type A headless screws. The pull-out strength for Type B headless screws was similar to the Type A headless screws and the stiffness was around 75% higher than for the Type A headless screws.

FIG. 8 shows the maximum push-out strength and stiffness for Type A, Type B and Type C square headed screws. FIG. 9 shows the maximum push-out strength and stiffness for Type A, Type B and Type C headless screws. Referring to FIGS. 8 and 9, the square headed screws recorded slightly higher push-out properties than the headless screws. The push-out stiffness values for the composite screws (Type B and Type C) both with and without heads were approximately twice those obtained for the Type A screws (˜2.8 kN·mm⁻¹). The maximum push-out loads for the Type B and Type C screws were approximately 70% higher than for the Type A screws.

The maximum shear load and stiffness for Type A, Type B and Type C screws were measured using a modified double shear test according to the standard BS 2782-3:Methods 340A and 340B:1978.

The testing arrangements for the double shear test were as follows. The crosshead speed of the machine was 5 mm/min and the load cell capacity was 5 kN. The maximum shear load and stiffness were determined as the maximum load recorded during the test and the maximum gradient in a load-deflection plot. The measurements were carried out in triplicate (n=3).

The results of the double shear tests are shown in FIG. 10. No significant difference between the maximum shear load and shear stiffness for Type B and Type C screws was seen. The composite screws (Type B and Type C) recorded an increase of approximately 30% and approximately 40% in the maximum shear load and stiffness in comparison with the Type A screws.

The maximum torque, stiffness and breaking angle were measured according to the standard ASTM F 2502-05 using a torque arrangement attached to an Instron 3367. The tensile force was converted into torque by using a rotating wheel mounted on a bearing stand. A calibration was performed in order to convert the deflection into rotation angle. The crosshead speed of the machine was 5 mm/sec, which equated to 1 revolution per minute (RPM) according to the calibration and the load cell capacity was 33 kN. According to the standard, the gauge length should be 20% (˜6 mm) of the total thread length as the screws were fully threaded. The maximum torque is represented by the highest recorded value of the torque during the test and the breaking angle was the rotation angle at the maximum torque. The measurements were carried out in triplicate (n=3).

FIG. 11 shows the torsional properties (maximum torque, breaking angle and stiffness) for the Type A, Type B and Type C screws. The maximum torque values for the composite screws (Type B and Type C) were slightly lower than for the Type A screws (˜1000 mN·m). The breaking angles for the two composite screws (Type B and Type C) were similar, but around half of that for the Type A screws. Torsional stiffness for the Type C and Type B screws were ˜100% and ˜50% higher than that for the Type A screws (˜100 mN·deg⁻¹) respectively.

The failure modes for the Type A, Type B and Type C screws observed in each of the mechanical tests are summarised in Table 2 below.

Neither natural bone nor synthetic bone models were used in the applicant's experiments. Metallic tools were applied in order to determine the optimum mechanical properties of the screws. Further, this avoided any possible variability that could have occurred if bone model materials had been used during the tests.

Fractured screws during the flexural test were conducted for SEM investigation. Cross-sections of the screws were sputter-coated with platinum and examined using a JEOL 6400 SEM with an accelerating voltage of 15 kV in secondary electron mode (SE).

FIG. 12 shows three such SEM micrographs. The left hand micrograph shows a fracture surface of a Type A screw after flexural test. The middle micrograph shows a fracture surface of a Type C screw after flexural test. The right hand micrograph shows a fracture surface of a Type B screw after flexural test.

Tables 3 and 4 below show some of the measured mechanical properties of the screws under test.

TABLE 3

| | | Flexural | | Shear | Pull-out |
|---|---|---|---|---|---|
| | Maximum | stiffness | Maximum | stiffness | stiffness |
| | flexural | (N · | shear load | (kN · | (kN · |
| Screw | load (N) | mm⁻¹) | (kN) | mm⁻¹) | mm⁻¹) |
| Type A | 190 ± 19 | 211 ± 8 | 2.20 ± 0.03 | 2.1 ± 0.1 | 2.2 ± 0.1 |
| Type B | 308 ± 10 | 393 ± 15 | 2.6 ± 0.2 | 3.5 ± 0.3 | 3.80 ± 0.06 |
| Type C | 390 ± 27 | 352 ± 17 | 2.9 ± 0.2 | 3.3 ± 0.1 | 4.9 ± 0.2 |

TABLE 4

| | Dimensions (mm) | | | | | |
|---|---|---|---|---|---|---|
| | Outer | Core | | | | Maximum |
| | diam- | diam- | | | Pull-out | torque |
| Screw | eter | eter | Pitch | Length | strength (kN) | (mN · m) |
| Type A | 6 | 4.75 | 1.25 | 32 | 0.87 ± 0.1 | 1006 ± 8 |
| square head | | | | | | |
| Type A | 6 | 4.75 | 1.25 | 28 | 0.9 ± 0.05 | 1006 ± 8 |
| headless | | | | | | |
| Type C | 6 | 4.75 | 1.25 | 32 | 0.55 ± 0.1 | 881 ± 66 |
| square head | | | | | | |
| Type C | 6 | 4.75 | 1.25 | 28 | 1.6 ± 0.3 | 881 ± 66 |
| headless | | | | | | |
| Type B | 6 | 4.75 | 1.25 | 32 | 0.42 ± 0.2 | 876 ± 180 |
| square head | | | | | | |
| Type B | 6 | 4.75 | 1.25 | 28 | 0.91 ± 0.2 | 876 ± 180 |
| headless | | | | | | |

As noted previously, a plate and screw system is commonly utilised for treatment of internal bone fractures. In use, mechanical failure of this combination may occur in the bone (stripping the bone thread), in the screw (screw fracture) or in the plate, which can cause non-union and malunion of fractures. The mode of failure depends on the mechanical characteristics of the screws and plate and the screw thread design.

The holding power and pull-out strength may represent the maximum tensile strength recorded for pulling the screws out of the bone or screw failure. The performance of screws during bone fixation may be controlled by different parameters such as thread depth, length, shape, surface finish and the mechanical properties of the screw materials. As the screw depth increases, the proportion of engaged bone with the thread and thus the holding power also increases. The screw length should have a similar effect to that of the thread depth. Sharp screw threads can cause cracks within the bone during insertion and consequently decrease the pull-out strength. However, a rough thread surface could increase the interlock with the bone and accordingly improve the holding power; however, a higher insertion torque would probably be required. Implants, e.g. fixation devices, with mechanical properties closer to bone could prevent the stress concentration on the bone and thus increase the required load to the failure.

Generally, it may be desirable to minimise or avoid buckling of fibres during manufacture of an implant, e.g. a fixation device such as a screw. Thus, for example, it may be preferred to reduce, minimise or eliminate the amount of axial pressure applied when forming a screw head. Alternatively or additionally, the precursor could be designed in such a way that the or a portion of the precursor that may be compressed axially during manufacture, e.g. in order to form a screw head, may contain few, if any, fibres that are oriented such that they could be buckled.

Strengths for Type A screws with and without heads were similar, as the failure modes did not change, whilst pull-out strength for Type C and Type B headless screws increased by ˜200% and 100% in comparison with the screws with heads. During the push-out test, square headed and headless screws failed via a buckling mode (see Table 2) and the strengths for square headed and headless Type B and Type C screws were similar and higher than for Type A screws.

In general, and as would be expected, the composite (Type B and Type C) screws exhibited significantly better mechanical properties than the Type A screws.

During a pull-out test or fixation of bone fracture, the screws can fail by two routes; screw failure or bone failure. Screw failure can occur via two modes; screw fracture (fracture at the cross-section) or thread failure due to shear. Bone failure occurs through shearing of the bone. The maximum force that can be endured by the screw before failure (pull-out strength) depends on the mode of failure. It is possible to predict the maximum load accompanying each mode of failure by using the following equations:

Screw fracture; Fₘₐₓ=A_{c}σₛₜ Equation 1

Thread failure; Fₘₐₓ=Aₛσₛₛ Equation 2

Bone failure; Fₘₐₓ=A_{b}σ_{sb} Equation 3

where σₛₜ, σₛₛ and σ_{sb} are the tensile strength for the screw material, the shear strength for the screw material and the shear strength for the bone, respectively. A_{c}, Aₛ and A_{b} are the minimum cross-sectional area of the screw (core), the shear area for external thread (screw thread) and internal thread (tapped bone thread), respectively.

In the applicant's experiments, the failure mode for all screws (Type A, Type B and Type C) was screw fracture at the core diameter. This was as expected, based on the application of the equations above for the dimensions of the screws under test. This was due to the tensile stress area being greater than the cross-sectional area for the screws.

The Type B and Type C screws were manufactured to ensure that the fibres incorporated also reinforced the thread, which can be seen in the x-ray photographs of the screws (see FIG. 4). The threads of the composite (Type B and Type C) screws were whitish, indicating high density components (i.e. glass fibres). Based on previous calculations, the failure mode could be controlled by the thread design. For the screws used in this study, the failure load of the thread was high even if the thread was completely PLA, i.e. a Type A screw, (˜3 kN pull-out load).

Since the screws may tend to fail at the core diameter rather than at the thread, it may be advantageous to include a higher density of reinforcement, e.g. fibres and/or particles, in the core of the screw shaft relative to the density of reinforcement in the threads.

The mechanical properties of screws depend mainly on their dimensions and the materials from which they are made. Typically, metallic screws may be stronger than bioresorbable ones. When metallic screws are used to fix bone fractures, failure may occur in the bone before the screw, whilst when bioresorbable screws are used failed may occur in the screw before the bone.

In the applicant's experiments, the flexural and shear properties for the composite (Type B and Type C) screws were superior to those of the PLA (Type A) screws. This is due to the reinforcement effect of phosphate glass fibres. Furthermore, it may be noted that the composite (Type B and Type C) screws demonstrated ductile behaviour (see Table 2). This may be advantageous for bone fixation devices, since it may prevent sudden failure occurring during the healing period.

Pull-out strength for the composite (Type B and Type C) headless screws in the applicant's experiments were higher than most of the reported data for metallic and bioresorbable screws when taking into account the different dimensions. Torque results were higher than for known bioresorbable screws and comparable with known metallic screws.

Generally, the mechanical data obtained for the three types of screw compare favourably with known commercially available resorbable screws. Mechanical properties for Type A, Type B and Type C screws (6 mm outer diameter) were higher than reported previously for resorbable screws. It is postulated that this may be a result of the forging manufacturing process. Also, this may be a consequence of the reinforcement effect of the fibres in the case of the Type B and Type C screws.

An in vitro degradation study of the three types of screw was performed according to the standard BS EN ISO 10993-13. Specimens of Type A (PLA), Type B (P40 UD/RM) and Type C (P40 UD) screws were placed individually into glass vials. The vials were filled with 50 ml of phosphate buffered saline (PBS) (pH=7.4±0.2) solution and maintained at 37° C. At various time points, the samples were extracted and blot dried before weighing. The samples were placed back into vials containing fresh PBS solution. Three replicates of each specimen type were measured and the average reported.

The percentage wet mass change (M_{w}), mass loss (M_{L}) and water uptake (W) were determined using the following equations:

$${M}_{W} = ( \frac{m - {m}_{i}}{{m}_{t}} ) \times 100 ( \% )$$

$${M}_{L} = ( \frac{{m}_{d} - {m}_{i}}{{m}_{t}} ) \times 100 ( \% )$$

$$W = ( \frac{m - {m}_{d}}{{m}_{d}} ) \times 100 ( \% )$$

where m is the mass of degraded sample measured at time t, mᵢ is the initial mass of the sample and m_{d} is the mass of the degraded sample after drying at 50° C. for four days.

Wet mass change against time for Type A (PLA), Type B (P40 UD/RM) and Type C (P40 UD(screws during degradation in PBS at 37° C. is shown in FIG. 13. The wet mass change showed a rapid increase during the first week to ˜0.9% for Type A screws and ˜0.7% for composite (Type B and Type C) screws. After 7 days the wet mass profile for PLA screws remained constant whereas the composite screws decreased gradually to ˜0.2% towards the end of the study (42 days). No significant difference in the wet mass was observed between Type B (P40 UD/RM) and Type C (P40 UD screws).

FIG. 14 shows the change in pH profile of PBS with degradation time for Type A (PLA), Type B (P40 UD/RM) and Type C (P40 UD) screws. No significant difference in pH between the different samples was seen during the degradation period. The pH remained constant at 7.5 for the duration of the study.

The percentage of water uptake against time for the samples investigated is shown in FIG. 15. The percentage of mass change against time for the samples investigated is shown in FIG. 16. Water uptake for Type A (PLA) screws remained constant at ˜0.8% and no mass loss was observed until 42 days. No significant difference in water uptake and mass loss was observed between the composite screws (Type B (P40 UD/RM) and Type C (P40 UD)). The water uptake and mass loss for the composite screws increased gradually to ˜1.25% and ˜1.1% respectively.

Bone plates and screws are used commonly for internal fixation of bone fragments after trauma. Screws can be used to fix plates to bone segments or to fix the fracture directly without plates depending on the type and location of the fracture. Metallic screws have been used for internal fixation but removal of the screws after bone healing in some cases is required, which may cause further trauma for the patient. A further problem associated with metallic screws is that even successful removal creates stress risers by leaving behind screw holes which can lead to re-fracture.

The screws, in particular the composite screws, manufactured and tested in the applicant's experiments could provide alternatives for metallic screws in order to eliminate a need for the removal process, as they are made from bioresorbable materials. These screws also contain phosphate glass fibres which have similar composition to natural bone. Phosphate glasses break down in the body into calcium and phosphate and are bioactive and ostoconductive.

Scotchford et al. (Scotchford C A, Shataheri M, Chen P S, Evans M, Parsons A J, Aitchison G A, et al. Repair of calvarial defects in rats by prefabricated, degradable, long fibre composite implants. J Biomed Mater Res A 2010 January; 96(1): 230-238) investigated in vivo study for PCL/PGF composite discs by using a rat animal model for 26 weeks. They observed lack of inflammatory mediators from the histological assessment and a significant increase in bone formation during the study. This could suggest that the screws manufactured and tested in the applicant's experiments should be acceptable for use in human or animal bodies.

The initial mechanical properties for the Type B and Type C screws were greater than for the Type A screws. Furthermore, the Type B and Type C screws in particular may have potentially acceptable mechanical properties for use in internal bone fixation.

In the case that it would be desirable to maintain the mechanical properties of the implants, e.g. fixation devices such as screws, for longer periods during degradation, the fibres and/or particles could be surface treated, e.g. using a coupling agent, in order to enhance the interface between the fibres and/or particles and the polymeric matrix. Factors to consider when selecting a suitable coupling agent include biocompatibility, resistance to water and the ability to interact chemically with the particles and/or fibres, e.g. PGF, and the polymeric matrix, e.g. PLA. Suitable coupling agents may include silanes (particularly with functional groups tailored to the polymer), phosphate functionalised organic materials such as bisphosphonates or functionalised oligomeric PLA, e.g. as described in US2012016475.

Controlling the interface between the matrix and the fibres and/or particles embedded therein may be crucial in producing fixation devices in accordance with the present invention, which retain their mechanical properties for extended periods of time within the human or animal body.

It will be appreciated that the method of manufacture may be used, e.g. with appropriate mould design, to produce fixation devices having many forms, e.g. fully threaded or partially threaded screws (which may be headless), pins, rods and plates, and dimensions.

Equally, it will be appreciated that the method of manufacture may be used to produce fixation devices having a wide range of compositions. For instance, such fixation devices may have a wide range of fibre or particle mass fractions and fibre or particle volume fractions.

Also, it will be appreciated that the apparatus and process methodology used may vary. For example, if the invention were scaled up, then a different, most likely more sophisticated apparatus may be used instead of the relatively simple, laboratory-scale apparatus described above and shown in the drawings.

The method of manufacture may be used to produce fixation devices containing other bioresorbable and/or biocompatible polymeric materials or matrices and/or other bioresorbable and/or biocompatible particles and/or fibres.

The method of manufacture may also allow for the distribution and arrangement of fibres and/or particles within the finished fixation device to be varied and/or controlled such that the mechanical properties of the finished fixation device may be such that the fixation device is particularly well suited for its intended use. For instance, a screw may be produced in which the threaded portion of the shaft is reinforced by particles and/or fibres to a greater or lesser extent than the core of the shaft.

1. A method of manufacture of an at least partially bioresorbable implant, the method comprising the steps of: providing an at least partially bioresorbable precursor containing a polymeric material; and forming an implant by forging the precursor.

2. A method according to claim 1, wherein forging is carried out at a temperature between the glass transition temperature (T_{g}) and the crystallisation temperature (T_{c}) for the polymeric material.

3. A method according to any one of the preceding claims, wherein the polymeric material is at least partially bioresorbable.

4. A method according to claim 4, wherein the polymeric material comprises one or more of poly lactic acid (PLA), poly glycolic acid (PGA) and polycaprolactone (PCL) or copolymers thereof.

5. A method according to any one of the preceding claims, wherein the precursor comprises a composite containing fibres and/or particles embedded in a polymeric matrix.

6. A method according to claim 5, wherein the fibres are randomly oriented within the precursor or the fibres are aligned in one or more directions.

7. A method according to claim 5 or claim 6, wherein the precursor comprises a first region or layer in which the fibres are aligned in one or more directions and a second region or layer in which the fibres are randomly oriented.

8. A method according to claim 5, claim 6 or claim 7, wherein at least some of the fibres and/or particles are bioresorbable.

9. A method according to any one of claims 5 to 8, wherein the fibres and/or particles comprise glass fibres and/or particles.

10. A method according to claim 9, wherein the fibres and/or particles comprise phosphate-based glass fibres and/or particles.

11. A method according to claim any one of claims 5 to 10, wherein the fibres and/or particles and the polymeric matrix are both bioresorbable.

12. A method according to any one of claims 5 to 11, wherein the particles and/or fibres comprise at least 10% and/or up to 60% by volume of the precursor and/or the fixation device.

13. A method according to any one of claims 5 to 12 comprising the step of surface treating the particles and/or fibres, e.g. with a coupling agent, prior to embedding the particles and/or fibres in the polymeric matrix.

14. A method according to any one of the preceding claims comprising the preliminary step of manufacturing or providing a master plate and cutting the precursor from the master plate.

15. A method according to any one of the preceding claims, wherein the precursor is provided in the form of a bar.

16. A method according to any one of the preceding claims, wherein the implant is a fixation device.

17. A method according to claim 16, wherein the fixation device has the form of a screw, e.g. a cortical screw or a cancellous screw, a pin, a rod or a plate.

18. A method according to claim 16, wherein the screw has a rough thread surface.

19. An at least partially bioresorbable forged implant containing a polymeric material.

20. A system for internal fixation of bone fragments in humans or animals comprising at least one at least partially bioresorbable forged implant containing a polymeric material.

21. A method of fixing a bone fracture comprising the use of an implant manufactured in accordance with any one of claims 1 to 18, a fixation device according to claim 19 or a system according to claim 20.

22. A method of manufacture of an implant substantially as described herein with reference to the accompanying drawings.

23. An implant substantially as described herein with reference to the accompanying drawings.
